(19) United States
(12) Patent Application Publication
(10) Pub. No.: US 2021/0322312 A1
(43) Pub. Date: Oct. 21, 2021

(54) CANNABIDIOL COMPOSITION AND METHODS THEREOF

(71) Applicant: Kirsten K. Shepard, Austin, TX (US)

(72) Inventor: Kirsten K. Shepard, Austin, TX (US)

(21) Appl. No.: 17/268,329

(22) PCT Filed: Aug. 15, 2019
(86) PCT No.: PCT/US2019/046672

§ 371 Date: Feb. 12, 2021

Related U.S. Application Data
(60) Provisional application No. 62/719,301, filed on Aug. 17, 2018.

(51) Int. Cl. A61K 9/06; A61K 31/05; A61K 9/00

(52) U.S. Cl. CPC A61K 9/06; A61K 9/0014; A61K 31/05

(57) ABSTRACT

The present invention relates to a composition comprising cannabidiol (CBD) and a pharmaceutically acceptable carrier thereof in the form of a cream. The present invention further relates to methods of treating pain or inflammation by administering a therapeutically effective amount of the composition of the present invention to a subject in need thereof. The present invention also relates to a kit comprising the cannabidiol composition and a method of making the cannabidiol composition.

### TECHNICAL FIELD

Embodiments generally relate to a cannabidiol (CBD) composition, a kit thereof, and/or a method thereof. More particularly, embodiments may relate to a CBD composition including CBD and a pharmaceutically acceptable carrier, a kit including a CBD composition, a method of using a CBD composition, and/or a method of making a CBD composition.

### BACKGROUND

CBD is a non-euphoriant phytocannabinoid believed to be an endocannabinoid modulator that can provide anti-inflammatory and analgesic effects (Russo, E. B. 2008. Cannabinoids in the management of difficult to treat pain. Therapeutics and Clinical Risk Management, 4(1): 245-259). For example, a combination of Δ⁹-tetrahydrocannabinol and CBD might treat neuropathic pain and other types of chronic pain (Xiong, W. et al. 2012. Cannabinoids suppress inflammatory and neuropathic pain by targeting α3 glycine receptors. The Journal of Experimental Medicine, 209(6): 1121-1134; Iffland, K. and Grotenhermen, F. 2017. An Update on Safety and Side Effects of Cannabidiol: A Review on Clinical Data and Relevant Animal Studies. Cannabis and Cannabinoid Research, 2.1: 139-154). Recently a 1% to 10% CBD gel was tested transdermally in a rat model of arthritis using dosages of 0.6, 3.1, 6.2, and 62.3 mg CBD/day, wherein doses below 3.1 mg CBD/day did not alter the synovial membrane and wherein optimization was determined to be at 6.2 CBD/day (Hammel, D. C. et al. 2016. Transdermal cannabidiol reduces inflammation and pain-related behaviors in a rat model of arthritis. European Journal of Pain, 20(6): 936-948). Meanwhile, a CBD isolate (a CBD extract having 95% to 99.8% CBD) may be prepared from industrial hemp and used in a formulation of 1% to 50% CBD isolate (Martinez et al., W02016153347A1). Thus, there is considerable room for improvement to provide a CBD composition for use as a medicament.

### DETAILED DESCRIPTION

Embodiments may involve a composition including CBD (and/or a salt thereof) together with a pharmaceutically acceptable carrier. CBD is reproduced below as the compound of Formula I:

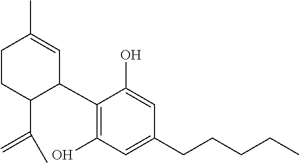 [SMILES: C=C(C)C1CCC(C)=CC1c1c(O)cc(CCCCC)cc1O]

CBD may be in the form of a CBD isolate and/or a CBD tincture having a relatively high amount of CBD. A CBD isolate and/or a CBD tincture may include, for example, at least about 95% CBD, more preferably at least about 99% (99+%) CBD. In one example, a CBD isolate may include about 99.05% CBD and one or more other non-psychoactive cannabinoids (e.g., cannabidivarin (CBDV) in an amount of about 0.14%). A CBD isolate may be readily prepared from industrial hemp or purchased in crystalline form (e.g., anhydrous CBD crystalline isolate). Meanwhile, a CBD tincture may include CBD dissolved in an alcohol (e.g., isopropyl alcohol, etc.). A CBD tincture may include, for example, CBD suspended in an oil solution (e.g., CBD oil). In this regard, a CBD isolate may be readily purchased or prepared from CBD oil (e.g., “pure” CBD oil) and/or from a hemp plant (e.g., “whole plant” CBD oil).

It should be understood that percentages discussed herein with regard to a CBD composition relate to a weight by weight (w/w) basis for illustrative purposes, which is the proportion of a particular substance within a mixture as measured by weight or mass. Thus, for example, a percentage a CBD tincture may be readily determined on a w/w basis using a density value of the CBD tincture. A CBD composition may include less than 1.0% CBD (e.g., less than 1.0% CBD isolate), preferably less than about .5% CBD (e.g., less than 0.5% CBD isolate). For example, a CBD composition may include a ratio of CBD to carrier between about .1 g to 5 g CBD and about 27 g to 1135 g carrier (between about 0.37% CBD and about 0.44% CBD). In one example, a CBD formulation may include 4.4 mg CBD/1.0 g CBD composition. Thus, particular dosages (e.g., effective amounts), number, and frequency of administration can be determined according to embodiments. For example, CBD to be applied to skin may be adjusted and/or determined according to embodiments, e.g., to eliminate or reduce adverse reactions, depending on the health of a subject in need thereof receiving treatment, depending on a particular disease being targeted, etc.

A pharmaceutically acceptable carrier may be a non-toxic carrier, a physiologically acceptable carrier, and so on. A pharmaceutically acceptable carrier may be in the form of an emulsion, a paste, a cream, a lotion, a gel, jelly, an ointment, an oil, an aerosol, a powder, a solvent, a liposome, a micelle, a peptide (e.g., albumin), a synthetic polymer (e.g., polyethylene glycol), a natural polymer (e.g., hyaluronic acid, dextran, chitosan), an n-dimensional material (where n=0, 1, 2, 3) such as, for example, a 0-dimensional nanomaterial (e.g., quantum dot, nanoparticle), a 1-dimensional nanomaterial (e.g., a nanotube, a nanorod), a 2-dimensional nanomaterial (e.g., a quantum well, a film), a 3-dimensional material, such as a matrix (e.g., polymeric matrix such as polyethylene glycol (PEG)), and so on. A pharmaceutically acceptable carrier may provide timed release, modulate the pharmacokinetic properties (e.g., absorption, distribution, metabolism, excretion) of CBD, modulate the pharmacodynamic properties (e.g., concentration at the site of action, resulting effect) of CBD, and so on. For example, a pharmaceutically acceptable carrier may be a topical composition that facilitates the transport of CBD across skin. In one example, a pharmaceutically acceptable carrier is preferably Lipopen Ultra Cream/LS Ultra Cream (Fagron, Inc.).

A CBD composition may further include one or more other optional therapeutic compounds. For example, a CBD composition may optionally include an essential oil. In one example, an essential oil includes Lavandula angustifolia, Citrus limon, Bowellia carterii Eucalyptus globulus, Arnica montana, E. smithii, E. radiata, Melaleuca alternifolia, Mentha piperita, (Lavender, Lemon, Frankincense, Eucalyotus, Arnica, Tea Tree and Peppermint, etc.), Ylang Ylang, Jasmine, Bees Wax, Mango Oil, Jojoba Oil, and so on.

Embodiments may involve a kit including a container to hold a CBD composition. A container may be formed of any suitable material such as, for example, a metal material, a polymeric material (e.g., plastic), etc. A container may include a dispenser portion, such as an opening that is accessed via a removable cap or a nozzle (e.g., a threaded cap, a latch cap, etc.), a removable film (e.g., a patch film), a perforated surface (e.g., a package perforated surface), and so on. Thus, a container may include a chamber to maintain a CBD composition, which may be accessed via the dispenser portion of the container for repeated use or for single use.

A container may be associated with an instruction regarding a CBD composition, such as a storage instruction (e.g., a storage condition), a use instruction (e.g., administration regimen, implant process), a disposal instruction, a warning, and so on. A container may also be associated with information regarding a CBD composition, such as a chemical formula, a structural formula, a property (e.g., molecular weight, melting point, concentration, etc.), an expiration date, and so on. In one example, a container may include a label to provide an instruction and/or information regarding a CBD composition, which may also be accessible from data storage such (e.g., computer server, computer readable medium, a database structure, etc.) in any data format (e.g., a text editor format (RTF), an image format (JPEG), a portable document format (PDF), a markup language format (HTTP, XML), a spreadsheet format, etc.). In one example, a container may be a transdermal patch, a bottle, a vial, and so on. In this regard, a label may be physically attached or otherwise associated with a container to provide instructions related to, e.g., an administration regimen. In another example, a container may be a laboratory storage container, a transport container, and so on. In this regard, a label may be physically attached or otherwise associated with a container to provide instructions related to, e.g., suitable storage conditions (e.g., pressure, temperature), hazard warnings, and so on.

Embodiments may involve a method including administering a CBD composition to a subject. The subject may include, for example, an animal, a human, and so on. The subject may be in need of a CBD composition. Accordingly, a method may include identifying a subject in need of a CBD composition to relieve pain, to relieve inflammation, and so on. A method may also include identifying a subject in need of a CBD composition that is susceptible to pain, to inflammation, and so on. In one example, a subject in need of a CBD composition may include a subject with arthritis, a subject with a muscle injury, a subject with a tissue injury, a subject with a joint injury, a subject with a ligament injury, a subject with a membrane injury, a subject with inflammation, and so on. In another example, a subject in need of a CBD composition may include an elderly subject, a subject involved in physically strenuous activity (e.g., athlete, etc.), a subject having a family history of inflammation, a subject having a pro-inflammatory biomarker (e.g., genetic, blood, etc.) profile, and so on. Thus, a method may include administering a CBD composition in response to such an identification of a subject.

A method may include administration of a CBD composition in an effective amount to elicit a biological or medicinal response in a tissue, in a system, in an animal, in a human, and so on. For example, an effective amount may include a therapeutically effective amount for the alleviation of one or more symptoms of a disease, a condition treated, and so on. In addition, an effective amount may include a prophylactically effective amount for a reduction of a severity and/or likelihood of one or more symptoms of a disease or condition. For example, a CBD composition may be administered in an effective amount to reduce pain and/or inflammation, to prevent pain and/or inflammation, to eliminate pain and/or inflammation, and so on. Thus, embodiments may involve a CBD composition for use as a medicament, a CBD composition for use in the treatment of pain, in the treatment of inflammation, and so on.

A method of administering a CBD composition may include applying a dose between about 2.2 mg CBD and about 4.4 mg CBD topically anywhere on skin to relieve pain and/or inflammation. For example, a method of administering a CBD composition may include applying about 0.5 g of a formulation of about 4.4 mg CBD/1.0 g CBD composition anywhere on a leg once daily to deposit a dose of about 2.2 mg CBD for relieving ankle pain and/or inflammation (although a CBD composition may be applied on an ankle to relieve ankle pain, etc.). Similarly, applying about 1.0 g of a formulation of about 4.4 mg CBD/1.0 g CBD composition to skin to deposit a dose of about 4.4 mg CBD may locally and/or systemically treat pain, inflammation, etc. A method of administering a CBD composition may also include rubbing a CBD composition onto skin after application. A method may further include a plurality of applications up to a daily CBD limit.

A CBD composition according to embodiments provides relatively improved pain-relief and anti-inflammatory response when applied topically on human skin. Unexpectedly, a CBD composition including less than 1.0% CBD, and in particular between about 0.22% (about 0.5 g of a formulation of about 4.4 mg/1.0 g CDB composition) and about 0.44% CBD (about 1.0 g of a formulation of about 4.4 mg/1.0 g CDB composition) is an effective amount to relieve neck pain after about 15-20 minutes post application in a retired athlete/coach (e.g., 1.0 g), to reduce swelling post plastic surgery (e.g., 0.5 g), to eliminate foot pain and knee pain in a homemaker (e.g., 0.5 g), etc. Notably, one subject with neck and shoulder pain that tried other topical formulations exhibited marked improved results in terms of time of effectiveness, duration of pain relief, and quality of pain relief (e.g., using a first application of 0.25 g of the same and a second application of 0.5 g 10 min later of the same).

Embodiments may involve a method of making a CBD composition. In one example, a method may include mixing CBD and a pharmaceutically acceptable carrier in the form of a cream to make a CBD composition including between about 0.1% and about 0.9% CBD. For example, a method of making a CBD composition may include using a CBD isolate to provide between about 0.2% and about 0.5% CBD in a CBD composition. In one example, CBD is in the form of a CBD isolate including at least 99% pure CBD. Thus, for example, a CDB composition may be produced as a formulation of 4.4 mg CBD/1.0 g CBD composition by mixing 4.4 mg CBD isolate (at least 99% pure) with 0.9956 g Lipopen Ultra Cream at room temperature using a Faragon Lab Basic Mixer in an 8 oz plastic container. Scale-up processes may be implemented, wherein a stock CBD composition is produced and diluted to provide a working CBD composition including between about 0.1% and about 0.9% CBD. A method of making a CBD composition may also include using a salt of CBD. Moreover, a method of making a CBD composition may include adding an optional therapeutic compound (e.g., an essential oil).

It should be understood that the indefinite articles “a” or “an” carry the meaning of “one or more” or “at least one”. In addition, as used in this application, a list of items joined by the terms “one or more of”, “at least one of” can mean any combination of the listed terms. For example, the phrases “one or more of A, B and C” and “one or more of A, B or C” can mean A; B; C; A and B; A and C; B and C; or A, B and C. Similarly, a list of terms joined by the term “and so on” or “etc.” can mean the list is not an exhaustive list and may be any combination of the listed terms. For example, the phrase “A, B, C, and so on” can mean A; B; C; A and B; A and C; B and C; or A, B and C.

Those skilled in the art will appreciate from the foregoing description that the broad techniques of the embodiments may be implemented in a variety of forms. Therefore, while the embodiments have been described in connection with particular examples thereof, the true scope of the embodiments should not be so limited since other modifications will become apparent to the skilled practitioner upon a study of the drawings and the specification described above and/or as follows.

1. A composition comprising between about 0.1% and about 0.9% cannabidiol (CBD) or a salt thereof, and a pharmaceutically acceptable carrier in the form of a cream.

2. (canceled)

3. The composition of claim 1, comprising between about 0.2% and about 0.5% CBD.

4. The composition of claim 3, comprising a formulation of about 4.4 mg CBD/1.0 g CBD composition.

5. The composition of claim 1, wherein the cream is Lipopen Ultra Cream.

6. The composition of claim 1, wherein the CBD is in the form of one or more a CBD isolate or a CBD tincture including at least 99% pure CBD.

7. The composition of claim 1, further including an essential oil.

8. A method comprising administering a CBD composition in an effective amount to treat one or more of pain or inflammation, where the CBD composition is to include between about 0.1% and about 0.9% CBD and a pharmaceutically acceptable carrier in the form of a cream.

9. (canceled)

10. (canceled)

11. (canceled)

12. The method of claim 8, wherein the administering includes applying the CBD composition anywhere on the skin to treat one or more of local pain, systemic pain, local inflammation, or systemic inflammation.

13. The method of claim 8, wherein the effective amount is a dose between about 2.2 mg CBD and about 4.4 mg to be applied once daily on the skin.

14. (canceled)

15. (canceled)

16. (canceled)

17. (canceled)

18. (canceled)

19. (canceled)

20. (canceled)

21. A method comprising mixing CBD or a salt thereof, and a pharmaceutically acceptable carrier in the form of a cream to make a CBD composition including between about 0.1% and about 0.9% CBD.

22. (canceled)

23. The method of claim 21, comprising between about 0.2% and about 0.5% CBD.

24. The method of claim 23, comprising a formulation of about 4.4 mg CBD/1.0 g CBD composition.

25. The method of claim 21, wherein the cream is Lipopen Ultra Cream.

26. The method of claim 21, wherein the CBD is in the form of one or more a CBD isolate or a CBD tincture including at least 99% pure CBD.

27. The method of claim 21, further including adding an essential oil.
